(19) United States
(12) Patent Application Publication
(10) Pub. No.: US 2003/0139802 A1
(43) Pub. Date: Jul. 24, 2003

(54) Medical device

(76) Inventors: Edward I. Wulfman, Woodinville, WA (US); Thomas J. Clement, Redmond, WA (US)

Correspondence Address: SPECKMAN LAW GROUP, 1501 WESTERN AVE, SUITE 100, SEATTLE, WA, 98101, US

(21) Appl. No.: 10/313,455

(22) Filed: Dec. 6, 2002

Related U.S. Application Data
(60) Provisional application No. 60/338,843, filed on Dec. 6, 2001.

(51) Int. Cl. A61F 2/06

(52) U.S. Cl. 623/1.15; 606/157

(57) ABSTRACT

The present invention provides a radially expandable device for use in the occlusion and repair of an undesired dilation in a vessel, such as an aneurysm, while maintaining flow both through the vessel and through branches of the vessel that may be located in proximity to the aneurysm. This is achieved by having a device with a differential pore size, wherein the portion of the device positioned in proximity to the aneurysm is of substantially smaller pore size than that portion of the device positioned away from the aneurysm.

### CROSS REFERENCE TO PRIORITY APPLICATION

This application claims priority to U.S. Patent Application 60/338,843 filed Dec. 6, 2001.

### TECHNICAL FIELD OF INVENTION

The present invention relates generally to implantable devices for therapeutic treatment of irregularities or defects in the vasculature and, more particularly, to a collapsible and expandable device capable of occluding the ostium of both axial and lateral aneurysms. The inventive devices are particularly well suited for the treatment of aneurysms located in proximity to one or more branches in the vasculature.

### BACKGROUND OF THE INVENTION

Irregularities in the vasculature or other structures, such as dilations producing aneurysms, account for a wide range of symptoms. Aneurysms pose a risk to health due to their potential for rupture, clotting, and/or dissection. For example, rupture of an aneurysm in the brain may cause stroke and potentially result in death, or produce neurological defects such as loss of sight, hearing or balance. Rupture of an aneurysm in the abdomen can lead to shock and other dangerous conditions. While a high fat diet, smoking and high blood pressure may contribute to a susceptibility for the development of aneurysms, recent studies indicate that the disease probably requires a basic genetic susceptibility that may be traceable to a single major locus, probably an autosomal dominant gene.

Aneurysms can occur at a variety of locations in a patient's vascular system. For example, an abdominal aortic aneurysm, a relatively common type of aneurysm, involves distension of the aorta. Cardiac aneurysms, which are bulges in a weakened ventricular wall, are typically caused by myocardial infarction. “Berry” aneurysms, known for their resemblance to a small berry, are small, saccular aneurysms of cerebral arteries. Aneurysms are classified as being either axial or lateral. Axial aneurysms generally involve the entire circumference of the vessel and cause a length of the vessel to balloon outward. Lateral aneurysms involve the distension of only one side of the vessel and typically form a sac-like recess.

Treatment of aneurysms is conventionally accomplished by direct surgical intervention. For example, abdominally located lateral aneurysms may be treated by installing a clamp around the base of the aneurysm to prevent communication of blood between the aneurysm and the lumen of the vessel, thereby reducing pressure on the aneurysm and causing it to shrink. Treatment of aneurysms within the brain may be accomplished using a number of invasive therapies. Open surgical techniques require cutting into the skull and lifting brain matter away from the aneurysm so that it may be accessed, clipped or sutured closed, and cut away. However, these techniques are risky, and generally reserved until deemed absolutely necessary due to the resultant high mortality rate and high chance of neurological defects caused by the operation itself.

Both the high risk and generally unsatisfactory results of open surgery on aneurysms have led researchers to develop minimally invasive techniques for treating aneurysms from inside the blood vessels. While stents are generally used to hold a vessel open and restore structural integrity to a vessel, thereby improving or restoring flow through a vessel, they have also been employed to occlude aneurysms. One problem with employing stents to occlude aneurysms, and in particular lateral aneurysms, within a vessel is that a significant surface area of the stent directly contacts the vessel wall. This can lead to tissue damage due to neointimal hyperplasia and development of stenosis. Furthermore, care must be taken to avoid blocking adjacent branches of the vessel with the stent.

U.S. Pat. No. 5,951,599 discloses an occlusion system for endovascular treatment of an aneurysm in which a stent having a cylindrical permeable portion and a second less permeable portion is placed with the second, less permeable portion overlying the neck of the aneurysm. The stent is a mesh-type cylinder that may be deployed and expanded at the site of the aneurysm. The stent may be coated or lined with a thromboresisting material, an antiangiogenetic material, or angiogenetic material or growth factors.

U.S. Pat. No. 6,093,199 discloses an intra-luminal device for treatment of body cavities and lumens that secures coils or other embolic devices placed within the aneurysm with a retainer element placed across the neck of the aneurysm. The retainer element is held in place with one or more anchoring elements. The retainer element may employ time-release medicines to enhance or prevent clot formation, cell growth, scar tissue formation, and the like.

U.S. Pat. No. 6,168,592 discloses an artificial occlusion kit for retaining occlusion devices, such as coils, at an occlusion site, such as an aneurysm. Various types of coils are disclosed for use as retaining devices.

U.S. Pat. No. 6,348,063 describes an implantable device having a deflecting element for deflecting and filtering the flow of embolic material flowing in the common carotid arteries (CCA) toward the internal carotid artery (ICA), into the external carotid artery (ECA). The anchoring member may be a stent or another tubular member.

U.S. Pat. No. 6,482,227 discloses a stent graft including a hollow stent having interconnected struts and including a graft material such as open cell foam.

Known devices for occluding and/or isolating an aneurysm often employ a continuous tubular stent-type device as an anchoring means. Over time, however, in many patients, contact between stents and stent-like anchoring devices, vessel walls and blood may promote restenosis and occlusion of the vessel in the area of the stent. There thus remains a need in the art for devices which may be effectively employed in the treatment of aneurysms, and in particular in the treatment of lateral aneurysms.

### SUMMARY OF THE INVENTION

The present invention provides a radially expandable device that can be employed for the occlusion and repair of an undesired irregularity in a vessel, such as a dilation or aneurysm, while maintaining flow both through the vessel and through branches of the vessel that may be located in proximity to the aneurysm. The inventive device has a structural portion and an opaque or substantially impermeable region having differential pore sizes, respectively, wherein the generally opaque or substantially impermeable portion of the device, which is positioned in proximity to the aneurysm when the device is deployed, is of substantially smaller pore size than the structural portion of the device positioned away from the aneurysm. This permits the flow of fluids through the structural portion of the device located away from the aneurysm, while preventing or reducing the passage of materials into and out from the aneurysm. While we refer to exemplary medical devices of the present invention in the context of their usefulness for treating aneurysms, it will be recognized that the use of device is not limited to this application, and it will be understood that the device may be used other applications involving irregularities in a vessel wall or other physiological structure.

In one embodiment, the medical device of the present invention comprises an expandable mesh having a comparatively small pore size in the region that is placed in proximity to an undesired dilation or discontinuity in a vessel, such as an aneurysm, referred to herein as the “opaque region”. The medical device also comprises one or more support members having a comparatively larger pore size providing structural support to the opaque region, and permitting the flow of fluid into and out from any vessel opening that it may contact, and permitting contact between fluid in a vessel and vessel walls.

In a related embodiment, the inventive device comprises a first expandable mesh of comparatively small pore size, referred to herein as an “opaque mesh,” or “substantially impermeable layer” that may be positioned in proximity to the mouth of the aneurysm when the device is deployed. The opaque mesh and/or substantially impermeable layer is mounted to and/or supported by a second, structural mesh of a sufficiently large pore size to permit the flow of fluids through the structural mesh. The structural mesh is preferably formed of expandable material and may be generally tubular in shape. In a preferred embodiment, the structural mesh forms an oncontinuous or discontinuous, generally tubular structure. The opaque mesh or substantially impermeable layer is of sufficiently small pore size to restrict the flow of fluid and particulate material into and out from the aneurysm. The opaque region and opaque mesh preferably having an average pore size of less than 1000 microns, more preferably less than 500 microns and, in some embodiments, less than 100 microns.

In one embodiment, the opaque mesh or opaque region that is positioned in proximity to the aneurysm is supported at each end by a structural member constructed to engage structural physiological elements in proximity to the aneurysm, such as vessel walls, upon deployment of the device. The structural member(s) may comprise a generally cylindrical, or at least partially generally cylindrical region of structural, expandable mesh that is shaped to fit within the structural physiological element(s) in proximity to an aneurysm, such as a blood vessel, forming a supporting ring positioned at each end of the opaque mesh or substantially impermeable layer. In this embodiment, the inventive device has the appearance of a saddle and the structural members, in combination with the opaque or substantially impermeable region, form a non-continuous, generally tubular and cylindrical structure. Each structural member preferably has a pore size sufficiently large to permit the flow of fluid through the member and contact between fluid flowing through a vessel and the interior vessel wall. Providing a device having discontinuous structural members positioned at or near the ends of an opaque mesh or opaque region rather than a continuous, relatively large, tubular structural mesh member reduces the area of contact between the structural mesh member(s) and the vessel wall, thereby reducing the risk of tissue damage and stenosis. It also allows effective placement of the aneurysm closure device in a wide variety of physiological settings where a continuous tubular device would be less effective, such as at or near branch points in blood vessels, and the like.

In one embodiment, one or more substantially impermeable layer(s) further occludes the flow of matter into, and out from the aneurysm. By substantially impermeable, we mean that the flow of fluids between the interior of the aneurysm and the interior of a blood vessel proximate the aneurysm, under physiological blood flow conditions, is less than 5 ml/hour, preferably less than 1 ml/hour and, in some embodiments, less than 0.1 ml/hour. In one embodiment, the substantially impermeable member may be a woven or non-woven fabric member. The substantially impermeable member(s) may be associated with one or more compositions, such as anti-bacterial, anti-microbial, anti-thrombogenic and anti-restenosis agents, which may be beneficially employed in conjunction with the inventive device. Such compositions are well known in the art, and means for associating such agents in a substantially impermeable member are also well known in the art.

The above-mentioned and additional features of the present invention and the manner of obtaining them will become apparent, and the invention will be best understood by reference to the following more detailed description, read in conjunction with the accompanying drawings.

### BRIEF DESCRIPTION OF THE DRAWINGS

FIG. 1 is a side view of a first embodiment of one embodiment of the inventive device.

FIG. 2 is a perspective view of the embodiment of the inventive device shown in FIG. 1.

FIG. 3 is a perspective, partially exploded view of a second embodiment of the inventive device.

FIG. 4 is a perspective, partially exploded view of a third embodiment of the inventive device.

FIG. 5 illustrates the placement of the device of FIG. 4 within a vessel.

FIG. 6 illustrates the placement of the device of FIG. 2 within a vessel.

### DETAILED DESCRIPTION OF THE INVENTION

As discussed above, the present invention provides a device for the occlusion of unwanted irregularities and dilations, such as aneurysms, in a vessel of the body, the device comprising a first region of relatively small pore size that is relatively impermeable to fluids and a second structural region of relatively larger pore size that is highly permeable to fluids. The inventive device may be implanted in the body on a temporary, permanent or semipermanent basis.

FIG. 1 shows a first occlusion device 10 of the present invention in a collapsed, or non-expanded, form for insertion into the body. Device 10, in its collapsed condition, is sized for introduction and guidance to the deployment site using an intravascular catheter and/or intravascular guidance and deployment system. Such intravascular guidance and deployment systems are well known in the art and are routinely used, for example, in the placement and deployment of stents. Device 10, in its expanded condition, is sized to fit within the desired vessel and to contact the inner vessel wall.

Device 10, which is generally tubular in shape, is formed of a first structural region of expandable mesh 12 of relatively larger pore size and a second region of expandable mesh 14 of relatively smaller pore size. “Generally tubular in shape” comprehends structures that have a continuously or discontinuously cylindrical configuration, as well as non-cylindrical configurations, such as oval, eccentric and other non-cylindrical and irregular, curved configurations. The overall exterior configuration of device 10 preferably corresponds generally to the configuration of the vessel or other physiological structure(s) where device 10 is intended to be placed.

The term “mesh” comprehends any structure having open spaces that are permeable to liquids and gases, and specifically comprehends net-like, screen-like and sieve-like structures, as well as porous structures. Examples of such porous materials include woven and perforated (including laser perforated) materials. The mesh structure(s) may have pores of substantially uniform or non-uniform size and/or shape. Stents having a variety of pore structures and shapes are well known in the art and may be adapted for use in the medical devices of the present invention.

Relatively smaller pore size region 14 may have a dimension, such as length, that is generally coextensive with a dimension, such as a length, of the relatively larger pore size structural region of the device. Alternatively, region 14 may be of a different dimension, generally a smaller dimension, than a corresponding dimension of structural member 12. Region 14 may be sized to generally cover the mouth of an irregularity, such as a vessel dilation, for example the ostium of an aneurysm, desired to be occluded. In general, region 14 is sized to extend for at least 20% of the length of structural member 12. In another embodiment, region 14 extends for at least 30% of the length of structural member 12 and, in yet another embodiment, region 14 extends for at least 50% of the length of structural member 12. In another embodiment, region 14 extends for no more than 50% of the length of structural member 12 and, in yet another embodiment, region 14 extends for no more than 25% of the length of structural member 12.

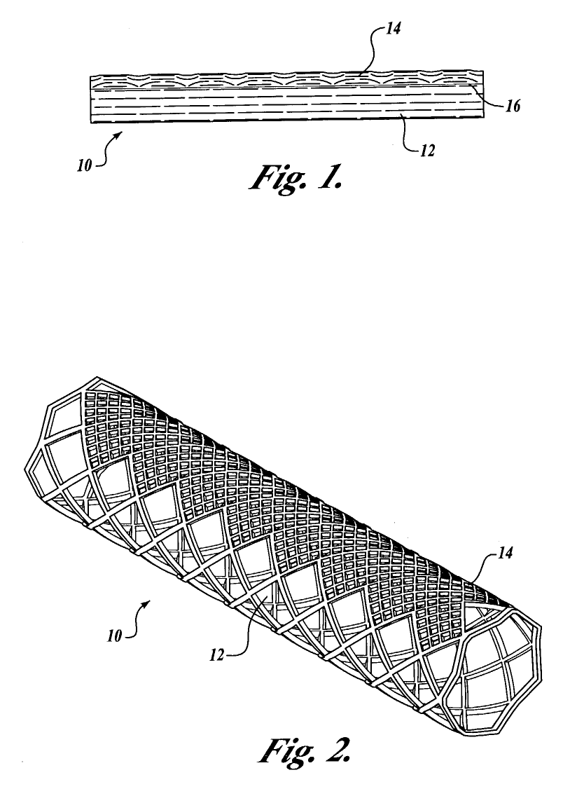

Smaller pore size region 14 is designed to extend over substantially the entire surface area of a vessel irregularity or dilation. Smaller pore size region generally extends over no more than 50% of the circumference of device 10 and, in some embodiments, extends over no more than 40% or 30% of the circumference of device 10. Although smaller pore size region 14 is shown in FIG. 2 as a rectangular region, it will be appreciated that other configurations may be used, and that multiple, separated smaller pore size regions 14 may be provided in a device 10.

Structural member 12 provides structural support and is generally permeable to fluids, while smaller pore size region 14 restricts the flow of fluids into and out from a vessel irregularity, such as an aneurysm. In the embodiment shown in FIG. 1, regions 12 and 14 may be formed from separate mesh components that are overlaid and permanently joined along their boundary. In another embodiment, regions 12 and 14 are formed from separate mesh components that are joined, in proximity to their boundaries, but do not entirely overlie one another.

FIG. 2 shows a device 10 in its expanded condition. Smaller pore size region 14 is placed in proximity to a vessel irregularity to prevent fluid exchange and transfer, and larger pore size structural region 12 contacts the interior vessel wall and provides secure placement of the medical device. Either or both mesh structure(s) may be impregnated, or coated, or otherwise associated, with one or more therapeutic agents, such as anti-bacterial, antimicrobial, anti-thrombogenic and anti-stenosis agents.

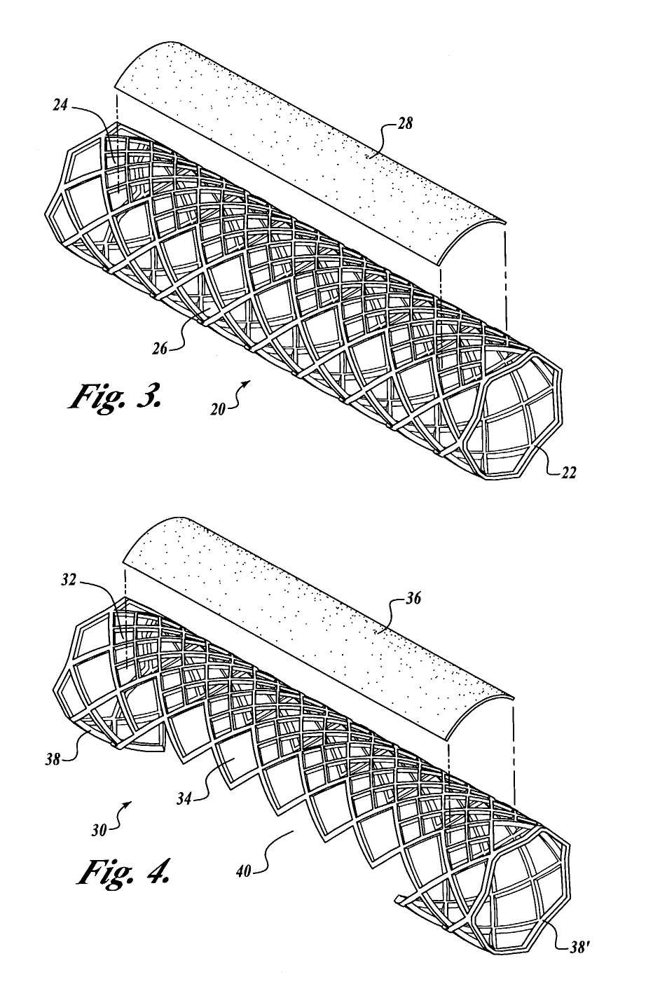

Another embodiment of the present invention is shown in FIG. 3. Occlusion device 20 comprises an expandable structural mesh portion 22 having a permeable region 26 of relatively large pore size. As with device 10, device 20 has a generally tubular shape and is sized to fit within a vessel and to contact the vessel wall when expanded. In the embodiment of FIG. 3, a layer 28 of substantially impermeable material is preferably positioned on an inner or intermediate or outer layer of the device an area where it will be near the vessel irregularity when the device is deployed. Substantially impermeable layer 28, which acts to further reduce the flow of fluid into and out from the aneurysm to be occluded, preferably has a pore size of less than 100 microns and is preferably constructed from a woven or non-woven expandable material or fabric, such as Dacron™, Goretex™, Teflon™ and flexible polyethylene terephthalate (PET). Other materials are known in the art and may also be used. Layer 28 may optionally be impregnated, or coated, or otherwise associated, with one or more therapeutic agents, such as anti-bacterial, anti-microbial, anti-thrombogenic and anti-restenosis agents.

Substantially impermeable layer 28 may be mounted or affixed directly to permeable region 26 of structural mesh portion 22, and structural mesh portion 22 may comprise mesh having a substantially uniform pore size. Alternatively, structural mesh portion 22 may comprise mesh portions having two or more different pore sizes. A smaller pore size region 24 may be provided, for example, for mounting and/or supporting substantially impermeable layer 28. Contacting or bonding or affixing substantially impermeable layer 28 to a smaller pore region 24 of device 10 generally provides more stable positioning, affixation and retention of layer 28. Layer 28 may contact or be mounted on or affixed to an inner or outer surface of device 20, such as at smaller pore region 24, or it may be positioned between multiple layers of device 20.

Although impermeable layer 28 is shown as a single piece, single layer element, it will be recognized that multiple substantially impermeable layers having the same or different configurations and the same or different compositions may be mounted on or affixed to different regions of device 20 to reduce the flow of fluids into and out from one or more vessel irregularities. Similarly, multiple layers of substantially impermeable layers that overlap one another may be provided in device 20. In one embodiment, a substantially impermeable layer 28 may be provided on the outer surface of device 20, in proximity to an aneurysm when the device is deployed, and another substantially impermeable layer may be provided in the inner surface of device 20 in the same area. One advantage of this configuration is that different therapeutic agents may be associated with the different substantially impermeable layers. Thus, for example, a clotting or stenosing agent may be associated with the substantially impermeable layer provided on the outer surface of the device in proximity to an aneurysm, while an antistenosis agent, or an anti-clotting agent, may be associated with the substantially impermeable layer provided on the inner surface of device 20 in proximity to the blood flow. Other therapeutic compositions, and combinations of such compositions, may also be used.

FIG. 4 shows yet a further embodiment of the present invention. As in the embodiments illustrated i n FIGS. 1-3, occlusion device 30 is provided with a substantially impermeable layer 36 of relatively small pore size which is placed in proximity to, and restricts fluid flow into and out of, the mouth of a vessel irregularity, such as an aneurysm. Support member 34 is formed of a mesh having relatively large pore size that is generally permeable to fluids. Rather than having a continuous generally tubular shape, as the illustrated in FIGS. 1-3, however, support region 34 has a noncontinuous or discontinuous generally tubular configuration. In the embodiment illustrated in FIG. 4, support region 34 comprises a central region 32 to which substantially impermeable layer 36 may be affixed or contact, and a pair of structural support rings 38 and 38′ positioned at either end of device 30 which contact inner wall 50 of a vessel 42 as shown in FIG. 5. Device 30 is thus generally saddle-like in shape, having a substantial recess area 40 where structural elements of the device, when deployed, do not contact the vessel wall. This design has a reduced risk of damaging the vessel wall, results in reduced contact between the support structure of device 30 and the vessel wall(s) and, thus, reduced risk of infection and stenosis, while providing support for the desired impermeable layer or structure and occlusion of the vessel irregularity.

The device of FIG. 4 is described as having a non-continuous or discontinuous generally tubular structure. FIG. 4 illustrates a device embodiment having a pair of ring-shaped support structures provided generally at the ends of the device. It will be recognized that other configurations of non-continuous or discontinuous generally tubular structures may be employed. More than two ring-shaped structures may be provided, and the ring-shaped structures may form complete rings, or incomplete rings. That is, the ring-shaped structures may not be continuous themselves, and they may not have the same conformation(s). In an alternative embodiment, continuous or non-continuous ring-shaped structures may be supported by a structure that traverses recess area 40, forming multiple recess areas 40. In preferred embodiments, recess area(s) 40 preferably comprise at least about 20% of the surface area of generally tubular device 30, and in other embodiments, recess area(s) 40 preferably comprise at least 30% or 40% of the surface area of generally tubular device 30.

Expandable mesh components of the medical device of the present invention may be constructed of non-self-expanding materials, wherein the device is expanded after placement in the vessel by means of, for example, an expansion balloon, or other means well known to those of skill in the art. The expandable mesh employed in the inventive devices thus may be formed from any of a variety of materials that may be collapsed and that expand radially when released. Such materials are well known to those of skill in the art, and include stainless steel, tantalum, gold, titanium, nickel-titanium, plastic materials and any combination thereof. The mesh may be self-expanding, such that the inventive devices automatically expand to their final diameter after insertion into the vessel and upon being subjected to expansion conditions, such as elevated temperature. For example, mesh components may be formed of a nickel titanium alloy, such as Nitinol™ (Memry Corp., Bethel, Conn.) which expands upon heating to body temperature.

In use, the occlusion devices of the present invention are delivered through a catheter or the like to the desired location in a patient's vascular system or in other vessels within the patient's body in a collapsed or non-expanded form, using well known techniques. Once the device is positioned in the desired location, it is expanded to contact and conform to the inner vessel wall.

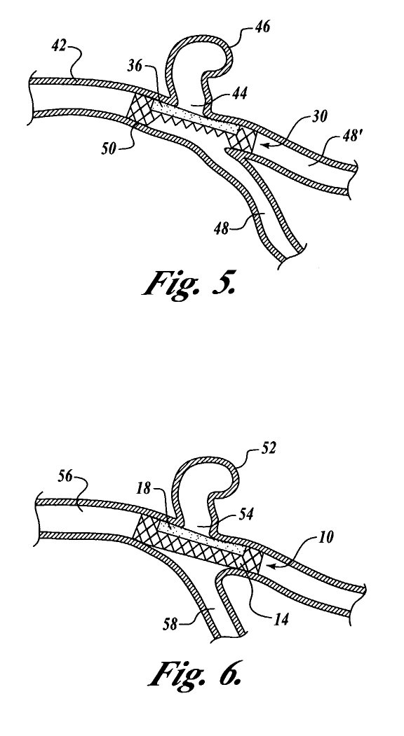

FIG. 5 illustrates the use of device 30 to occlude an aneurysm. Device 30 is positioned in vessel 42 in proximity to ostium 44 of aneurysm 46 such that the flow of fluid into and out of aneurysm 46 is restricted by substantially impermeable layer 36 and mesh region 32 (not shown), while the flow of fluid through vessel branches 48 and 48′ is unaffected. Due to the saddle-like shape of device 30, contact between the device and the inner wall 50 of vessel 42 is minimized. Use of device 20 to occlude aneurysm 52 in vessel 56 is illustrated in FIG. 6. Similar to the use of device 30 shown in FIG. 5, device 10 impedes the flow of fluid into and out of ostium 54 of aneurysm 52 by means of substantially impermeable layer 18 and mesh region 12 (not shown), while allowing the flow of fluid through vessel 56 and vessel branch 58.

While in the foregoing specification this invention has been described in relation to certain preferred embodiments, and many details have been set forth for purpose of illustration, it will be apparent to those skilled in the art that the invention is susceptible to additional embodiments and that certain of the details described herein may be varied considerably without departing from the basic principles of the invention.

We claim:

1. A device for occlusion of an irregularity in a vessel of a patient's body, comprising:
(a) a first structural mesh region having a pore size sufficiently large to permit flow of fluids through the structural mesh region and sized to fit within the vessel and to contact a vessel wall when expanded, the first structural mesh region having a discontinuous, generally tubular configuration; and
(b) a second region of smaller pore size provided in association with the structural mesh region, the second region having a surface area at least large enough to cover the irregularity in the vessel and having a pore size sufficiently small to impede flow of fluids into and out from the irregularity;
whereby flow of fluids through the vessel and through branches of the vessel located in proximity to the irregularity is maintained when the device is positioned in the vessel.

2. The device of claim 1, wherein the structural mesh region comprises at least two ring portions separated from one another by a recess.

3. The device of claim 2, wherein the recess area comprises at least 20% of the surface area of the device.

4. The device of claim 1, wherein the second region comprises an opaque mesh having an average pore size of less than 500 microns.

5. The device of claim 3, wherein the second region comprises an opaque mesh having an average pore size of less than 100 microns.

6. The device of claim 1, wherein the structural mesh region and the second region overlie one another.

7. The device of claim 1, wherein the structural mesh region is self-expanding under predetermined expansion conditions.

8. The device of claim 1, wherein the second region comprises a substantially impermeable layer having an average pore size of less than 100 microns.

9. The device of claim 8, comprising at least two spatially separated substantially impermeable layers.

10. The device of claim 9, wherein each of the at least two spatially separated substantially impermeable layers is associated with a therapeutic agent.

11. The device of claim 10, wherein each of the two spatially separated substantially impermeable layers is associated with a different therapeutic agent.

12. The device of claim 8, wherein the substantially impermeable layer is associated with a therapeutic agent.

13. The device of claim 1, wherein the second region comprises a substantially impermeable layer comprising a woven or non-woven fabric material.

14. The device of claim 1, further comprising at least two second regions of smaller pore size.

15. The device of claim 1, wherein the device is associated with at least one therapeutic agent.

16. The device of claim 15, wherein the therapeutic agent is selected from the group consisting of: anti-bacterial, anti-thrombogenic, anti-stenosis agents and combinations thereof.
